(19) United States
(12) Patent Application Publication
(10) Pub. No.: US 2009/0240197 A1
(43) Pub. Date: Sep. 24, 2009

(54) CENTRAL VENOUS CATHETERS AND RELATED EQUIPMENT

(75) Inventors: Kevin P. Cowan, Allison Park, PA (US); Eugene A. Gelblum, Pittsburgh, PA (US); Alan D. Hirschman, Glenshaw, PA (US); David M. Reilly, Gibsonia, PA (US); Edward J. Rhinehart, Monroeville, PA (US); Frederick W. Trombley, III, Gibsonia, PA (US)

Correspondence Address: GREGORY L BRADLEY;MEDRAD INC, ONE MEDRAD DRIVE, INDIANOLA, PA, 15051, US

(73) Assignee: Medrad, Inc., Indianola, PA (US)

(21) Appl. No.: 12/297,637

(22) PCT Filed: Apr. 19, 2007
(86) PCT No.: PCT/US07/66930

§ 371 Date: Oct. 17, 2008

Related U.S. Application Data
(60) Provisional application No. 60/794,051, filed on Apr. 21, 2006.

(51) Int. Cl. A61M 25/092; A61M 25/09; A61M 25/10

(52) U.S. Cl. 604/95.02; 604/528; 604/101.01

(57) ABSTRACT

Systems, devices, and methods towards improved diagnosis and therapy in connection with central venous catheters (including PICC lines). Included among the many improvements broadly contemplated herein are: arrangements via which a venous catheter can move passively into position in central venous circulation; arrangements via which a catheter can be actively guided without the use of a needle or guide wire; arrangements via which a catheter can be packaged to facilitate easy, rapid, and positionally accurate deployment by medical personnel while maintaining device sterility; and arrangements via which a catheter tip can be imaged during and after insertion.

### CROSS REFERENCE TO RELATED APPLICATION

This application claims the benefit of U.S. Provisional Application Ser. No. 60/794,051, filed on Apr. 21, 2006, the contents of which are incorporate herein by reference.

### FIELD OF THE INVENTION

The present invention relates generally to central venous catheters, e.g., as employed in patient care and cardiac CT angiography, and to equipment which relates to or provides support to the same.

### BACKGROUND OF THE INVENTION

The historical introduction of catheters provided a boon to numerous medical applications, especially in the sphere of cardiac applications, but room has continually existed for improvement in the structure and makeup of the catheters themselves as well as in the types of equipment and arrangements that might be used in conjunction with or in support of catheters.

Generally, perennial problems have presented themselves in terms of how effectively a catheter can be deployed in and along a patient's vein, and how this might impact not only the comfort of the patient but also any medical risk that could be presented to the patient as a result. For instance, poor catheter design has long been associated with problems relating to the excessive distention of a vein or even the partial or total occlusion of blood flow. While the field thus does not lack for efforts to render ongoing improvements, many shortcomings do still exist. Other problems and challenges have been encountered in connection with matters literally and figuratively external to the deployment of a catheter, such as how to physically measure the deployment of a catheter, how to keep it or a surrounding region sufficiently sterile, and how to locate a catheter tip once deployed into a patient.

By way of some specific catheter applications, the interior of coronary arteries and great vessels of the heart historically were imaged via X-Ray fluoroscopy, enhanced by injection of radio-opaque contrast fluids through arterial catheters. However, in recent years the introduction of multi-detector CT (MDCT) scanners has made it possible to create 3D images, nearly in real-time, not only of the inner lumen of great vessels and coronary arteries, but also of the anatomy of the surrounding cardiac structures.

A further advantage of MDCT is that the internal walls of the coronary vessels, structures surrounding the vessels, and calcification of the coronary vessel walls can be imaged, whereas coronary arteriography only delineates the internal lumen of those arteries. It has been demonstrated that coronary arteries can be imaged by injecting contrast fluid (alternatively, “contrast medium”) into the peripheral venous circulation, typically through a short catheter placed in an accessible vein of the arm or hand.

However, there are disadvantages inherent in coronary imaging via MDCT using the peripheral intravenous injection of contrast. Significant among these is the loss of image quality in view of the varying time delay and dilution of contrast as it travels the venous circulation to the heart and subsequently mixes with blood in the pulmonary circulation before reaching the arterial side of the heart. As such, U.S. Pat. No. 6,442,415 (Bis et al.) addresses the use of coronary CT angiography (CTA) by means of arterial injection of contrast into the aortic root. Although this method has been shown to produce outstanding images of the coronaries under MDCT, the procedure necessitates the introduction of an arterial catheter in the sterile environment of a cardiac catheterization lab, under the guidance of a skilled interventional cardiologist, radiologist or vascular surgeon.

A method that is less invasive than arterial catheterization of the heart, yet more invasive than an IV peripheral needle injection, is a central venous catheter approach. Here, the coronary arteries can be imaged by MDCT by introducing a catheter into the central venous circulation, preferably through the superior vena cava into the chambers of the right heart, or alternatively, into the cardiac venous circulation via the coronary sinus.

The injection of contrast here presents two distinct advantages over peripheral intravenous injection. First, the contrast arrives in the right heart in more concentrated form before its journey through the pulmonary circulation and then into the left heart, which in turn feeds both the arterial circulation and the coronary arteries. Second, the time delay and build-up of contrast fluid in the arm is eliminated when an intravenous injection is made through a peripheral vein in the arm. Normally such a build-up of contrast is sometimes responsible for excess image artifact and X-Ray scatter, especially in the right heart, which degrades the images of the heart and coronary arteries.

As a method for injecting fluids into the central venous circulation, central catheters have long been known. These are placed into veins in the chest or neck. These have been gradually replaced by PICC (peripherally inserted central catheter) technology. PICC lines are flexible catheters that are inserted typically through a vein in the arm into the central venous circulation near the heart. To aid in the catheter placement, a stiff needle or guide wire is provided in the lumen of the flexible catheter. Under the guidance of a fluoroscope (or an ultrasonic imaging device), the combination guide wire and catheter is typically threaded through the vessel into the central vena cava. Once in place, the needle or guide wire is removed, leaving the flexible catheter in place with the distal tip properly positioned for injection of fluid. These catheters can be left in place for days to months for the low flow-rate infusion of medication into the patient, and/or for sampling blood in patients with veins that have been compromised by disease or by the corrosive effects of chemotherapeutic drugs.

However, despite the advances hitherto made with PICC technology, there is tremendous room for improvement in the realm of injecting higher doses of contrast medium, at higher rates, into central venous circulation during the relatively short time-frame of a coronary angiographic procedure.

In view of the foregoing, numerous needs have been recognized in connection with developing and effecting improvements in the general sphere of coronary and venous imaging and particularly in connection with the equipment and methods employed.

### SUMMARY OF THE INVENTION

Broadly contemplated herein, in accordance with at least one presently preferred embodiment of the present invention, are systems, devices, and methods for improved diagnosis and therapy with central venous catheters and PICC lines.

Included among the many improvements broadly contemplated herein are:

arrangements via which the venous catheter can move passively into position in the central venous circulation;

arrangements via which a flexible venous catheter can be actively guided from the insertion point in a peripheral vein into the final location of the tip in the central venous circulation, without the use of a needle or guide wire;

arrangements via which a catheter can be packaged to facilitate easy, rapid, and positionally accurate deployment by medical personnel of average skill, while maintaining device sterility; and

Arrangements via which a catheter tip can be imaged during and after insertion.

In summary, there is broadly contemplated herein, in accordance with at least one presently preferred embodiment of the present invention, a catheter comprising: a catheter body; and an arrangement for promoting movement of the catheter body within a blood vessel; the arrangement for promoting movement comprising at least one entraining arrangement for entraining blood flow and urging the catheter body forward within a blood vessel.

Further, there is broadly contemplated herein, in accordance with at least one presently preferred embodiment of the present invention, a catheter comprising: a catheter body; and an arrangement for promoting movement of the catheter body within a blood vessel; the arrangement for promoting movement comprising at least one propulsion arrangement for applying a force from outside the catheter to urge the catheter body forward within a blood vessel.

Additionally, there is broadly contemplated herein, in accordance with at least one presently preferred embodiment of the present invention, a catheter comprising: a catheter body; and an arrangement for promoting movement of the catheter body within a blood vessel; the arrangement for promoting movement comprising an arrangement for assisting forward movement of the catheter body via physical engagement with a blood vessel.

Yet further, there is broadly contemplated herein, in accordance with at least one presently preferred embodiment of the present invention, a catheter comprising: a catheter body; and an arrangement for promoting movement of the catheter body within a blood vessel; the arrangement for promoting movement comprising a wound or braided portion for imparting increased flexibility to the catheter body.

Still further, there is broadly contemplated herein, in accordance with at least one presently preferred embodiment of the present invention, a catheter system comprising: a catheter body; and an arrangement for selectably varying a stiffness of the catheter body.

Additionally, there is broadly contemplated herein, in accordance with at least one presently preferred embodiment of the present invention, a catheter system comprising: a catheter body; and an arrangement for providing a propulsion force to promote movement of the catheter body within a blood vessel; the arrangement for providing a propulsion force comprising an arrangement for providing fluid to a blood vessel; the arrangement for providing fluid being configured to feed the catheter body to a blood vessel simultaneously with providing fluid to a blood vessel.

Furthermore, there is broadly contemplated herein, in accordance with at least one presently preferred embodiment of the present invention, a catheter system comprising: a catheter body; the catheter body comprising a plurality of minor conduits; a propulsion arrangement configured for selectively and separately providing fluid to each of the minor conduits to promote selective steering of the catheter body.

Moreover, there is broadly contemplated herein, in accordance with at least one presently preferred embodiment of the present invention, a catheter system comprising: a catheter; a sterile container which contains the catheter; the sterile container comprising an opening for surrounding an entry point on a patient's body.

Still additionally, there is broadly contemplated herein, in accordance with at least one presently preferred embodiment of the present invention, catheter system comprising: a catheter; a container which contains the catheter; and a measuring arrangement for measuring a length of the catheter which exits the container, at least a portion of the measuring arrangement being disposed on the container.

Furthermore, there is broadly contemplated herein, in accordance with at least one presently preferred embodiment of the present invention, a catheter measuring system comprising: a measuring wheel for measuring a length; and a cutting arrangement for cutting catheter; and a mounting arrangement which supports both the measuring wheel and the cutting arrangement.

Moreover, there is broadly contemplated herein, in accordance with at least one presently preferred embodiment of the present invention, a catheter body; an arrangement for facilitating location of the catheter body, the arrangement for facilitating location being disposed on the catheter body; and a tracking arrangement configured for tracking the arrangement for facilitating location when the catheter body is disposed within a patient's body.

Yet even further, there is broadly contemplated herein, in accordance with at least one presently preferred embodiment of the present invention, a method of manipulating a catheter, the method comprising: providing a catheter body; and selectably varying a stiffness of the catheter body.

Still even further, there is broadly contemplated herein, in accordance with at least one presently preferred embodiment of the present invention, a method of manipulating a catheter, the method comprising: providing a catheter body; providing a propulsion force to promote movement of the catheter body within a blood vessel; the step of providing a propulsion force comprising providing fluid to a blood vessel; and feeding the catheter body to a blood vessel simultaneously with providing fluid to a blood vessel.

Yet even additionally, there is broadly contemplated herein, in accordance with at least one presently preferred embodiment of the present invention, a method of manipulating a catheter, the method comprising: providing a catheter body, the catheter body comprising a plurality of minor conduits; selectively and separately providing fluid to each of the minor conduits to promote selective steering of the catheter body.

Still even additionally, there is broadly contemplated herein, in accordance with at least one presently preferred embodiment of the present invention, a method of manipulating a catheter, the method comprising: providing a sterile container which contains a catheter; surrounding and securing a portion of the sterile container about an entry point on a patient's body; and deploying the catheter into the patient's body.

Furthermore, there is broadly contemplated herein, in accordance with at least one presently preferred embodiment of the present invention, a method of manipulating a catheter, the method comprising: providing a sterile container which contains a catheter; and measuring a length of catheter which exits the container with a measuring arrangement at least partly disposed on the container.

Moreover, there is broadly contemplated herein, in accordance with at least one presently preferred embodiment of the present invention, a method of tracking catheter, the method comprising: providing a catheter body; disposing on the catheter body an arrangement for facilitating location of the catheter body; and tracking the arrangement for facilitating location when the catheter body is disposed within a patient's body.

The novel features which are considered characteristic of the present invention are set forth here below. The invention itself, however, both as to its construction and its method of operation, together with additional objects and advantages thereof, will be best understood from the following description of the specific embodiments when read and understood in connection with the accompanying drawings.

### BRIEF DESCRIPTION OF THE DRAWINGS

FIG. 1A illustrates a “passively guided” catheter of a first configuration.

FIG. 1B illustrates a “passively guided” catheter of a second configuration.

FIG. 1C illustrates a “passively guided” catheter of a third configuration.

FIG. 2A illustrates, in cross-sectional view, an “actively guided” catheter, involving reverse flow propulsion, of a first configuration.

FIG. 2B illustrates, in cross-sectional view, an “actively guided” catheter, involving reverse flow propulsion, of a second configuration.

FIG. 2C illustrates, in a radial cross-sectional view taken along line II-II, a portion of the catheter shown in FIG. 2B.

FIG. 2D illustrates, in cross-sectional view, an “actively guided” catheter, involving reverse flow propulsion, of a third configuration.

FIG. 3A illustrates, in cross-sectional view, an “actively guided” catheter, involving a divided outer annular chamber.

FIG. 3B illustrates, in a radial cross-sectional view, a portion of the catheter shown in FIG. 3A along with a schematically illustrated control element.

FIG. 3C illustrates a distal end of the catheter shown in FIG. 3A.

FIG. 4A illustrates an “actively guided” catheter involving deformable edges, in a first position.

FIG. 4B illustrates an “actively guided” catheter involving deformable edges, in a second position.

FIG. 5 illustrates an additional catheter embodiment involving reverse jet-flow.

FIG. 6A illustrates an “actively guided” catheter involving a “rotary millipede” configuration, in a first position.

FIG. 6B illustrates an “actively guided” catheter involving a “rotary millipede” configuration, in a second position.

FIG. 6C illustrates an “actively guided” catheter involving a “rotary millipede” configuration, in a third position.

FIG. 7 illustrates an “actively guided” catheter involving an “axial millipede” configuration.

FIG. 8 illustrates an “actively guided” catheter involving a “flow assisted” configuration.

FIG. 9 illustrates an “actively guided” catheter involving an “everting” configuration.

FIG. 10 illustrates an “actively guided” catheter involving a “variable stiffness” configuration.

FIG. 11A illustrates a an “actively guided” torque catheter with braiding.

FIGS. 11B and 11C respectively illustrate variant catheter tips that may be employed with the catheter of FIG. 11A.

FIG. 11D provides a close-up view of a catheter portion, with an alternative coiled component that could be employed in the catheter shown in FIG. 11A

FIG. 11E provides a close-up view of a catheter portion having cross-braiding as shown in FIG. 11A.

FIGS. 12A-12E show successive views of an “actively guided” catheter involving a double-balloon configuration, in various positions in a catheter insertion process.

FIG. 13A shows a catheter bag of a first configuration.

FIG. 13B shows a catheter bag of a second configuration.

FIG. 13C shows a catheter bag of a third configuration.

FIG. 14A shows a catheter dispensing arrangement.

FIG. 14B shows a measuring wheel for use in the dispensing arrangement of FIG. 14A.

FIG. 14C shows another measuring wheel configuration.

FIG. 15A shows a catheter arrangement which facilitates catheter tip location.

FIG. 15B shows a hand-held locator for use with the arrangement of FIG. 15A.

### DESCRIPTION OF THE PREFERRED EMBODIMENTS

Generally, improvements to catheters are contemplated herein in two general areas: “passively” guided catheters and “actively” guided catheters. The former tend to involve those catheters that are able to be deployed along a patient's vein either partly or wholly by virtue of any assistive forces provided by blood flow. The latter tend to involve those catheters that at least partly involve some external applied force to help deploy a catheter along a patient's vein. When deployed, catheters may deliver fluids such as saline solution, contrast solution and/or medication as variously discussed herebelow, or be used to withdraw and optionally redeliver fluids, for example blood, from the patient for testing or treatment purposes. If not otherwise stated herebelow, it should be understood that to the extent contrast delivery is discussed herebelow, medication delivery is also contemplated in the same posture, and vice versa.

Passively guided catheters are known, but are in great need of improvement. One conventional arrangement employing blood flow to guide catheters towards a heart is the “Swann-Ganz” balloon catheter. This device requires a separate lumen within the catheter, which allows the passage of air to inflate a balloon on the distal tip. As the catheter approaches the pulmonary artery, it is guided into the right atrium of the heart by the entrainment of blood flowing behind the balloon. A primary disadvantage of this catheter is that a dedicated arrangement for inflating the balloon is needed.

In accordance with a preferred embodiment of the present invention, there is broadly contemplated an arrangement wherein at the distal end of a catheter an entraining arrangement, for “capturing” blood flow to help “pull” the distal catheter tip (and by extension the entire catheter) along, is provided. More particularly, a dedicated arrangement for “inflating” or even just deploying such an entraining arrangement is not needed.

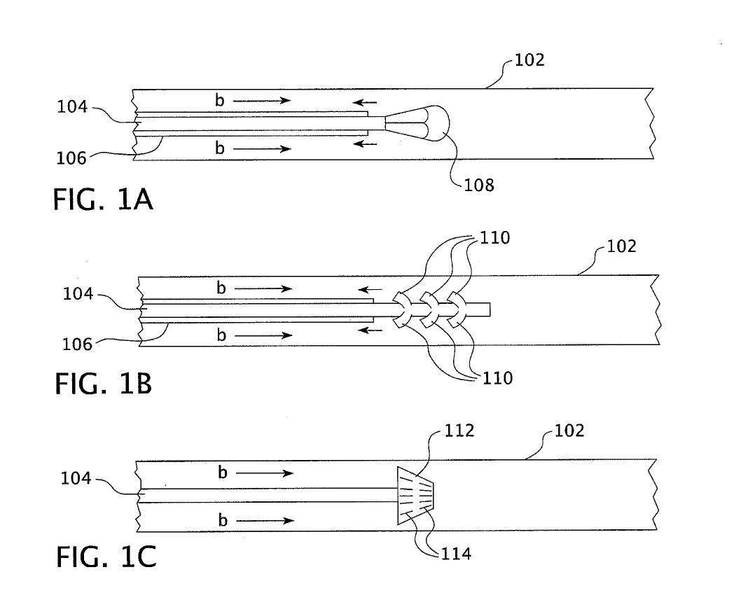

As shown in FIGS. 1A-1C, several arrangements in this posture are conceivable. FIG. 1A shows catheter 104 within walls of a blood vessel 102 where an outer sheath 106 can be retracted to permit a “parachute” 108 or analogous device (e.g., a polymer material with elastic or string segments extending therefrom) to release, and then entrain a portion of blood flow b. As can be appreciated, the force of blood flow will “pull” along the parachute 108 and hence catheter 104; this can be sufficient for propelling catheter 104 along the direction of blood flow b but could also conceivably be supplemented by an “active” propelling arrangement (conceivably one of those described in further detail here below).

FIG. 1B illustrates a variant embodiment where sheath 106 can be pulled back to expose wings or flaps 110 sufficient to “capture” and entrain blood flow. These can expand when the sheath is retracted and be caught by blood flow b and be forced outwardly away from the body of catheter 104. Alternatively, wings/flaps 110 can be essentially rigid and a sheath might not be needed.

FIG. 1C, for its part, illustrates yet another variant where a shroud 112 with perforations (114) “caps” distal end of catheter 104. Preferably, the “solid” surface area of shroud 112 will be sufficient as to entrain blood flow to a degree sufficient for pulling along the catheter 112, while perforations 114 may be of such a distribution and number as to reduce the risk of blood vessel occlusion and/or to act as a “damper” on the velocity with which the catheter 112 propagates along the blood vessel. While the use of an outer sheath may be desirable here, it may not be necessary.

By way of providing a further “assist” to the arrangements shown in FIGS. 1A-1C for “passively” propagating a catheter along a blood vessel, especially if a patient's blood flow may not be sufficiently strong to move the “entraining elements” (e.g., parachute, wings/flaps or shroud) forward, it is conceivable to pump saline solution into the blood vessel from an external source. The momentum of the injected saline solution will thus help drive the “entraining” elements and the catheter tip forward in the vessel. The injected saline solution also has the benefit of more fully filling or distending the blood vessel.

The disclosure now turns to a discussion of several “active” or “actively guided” catheters broadly contemplated in accordance with embodiments of the present invention. Here, catheters more or less rely upon force applied or urged from an external source (e.g., from the injection of saline solution) in order to propagate through a patient's blood vessel. Again, this would appear to be particularly favorable in the context of patients lacking strong blood flow.

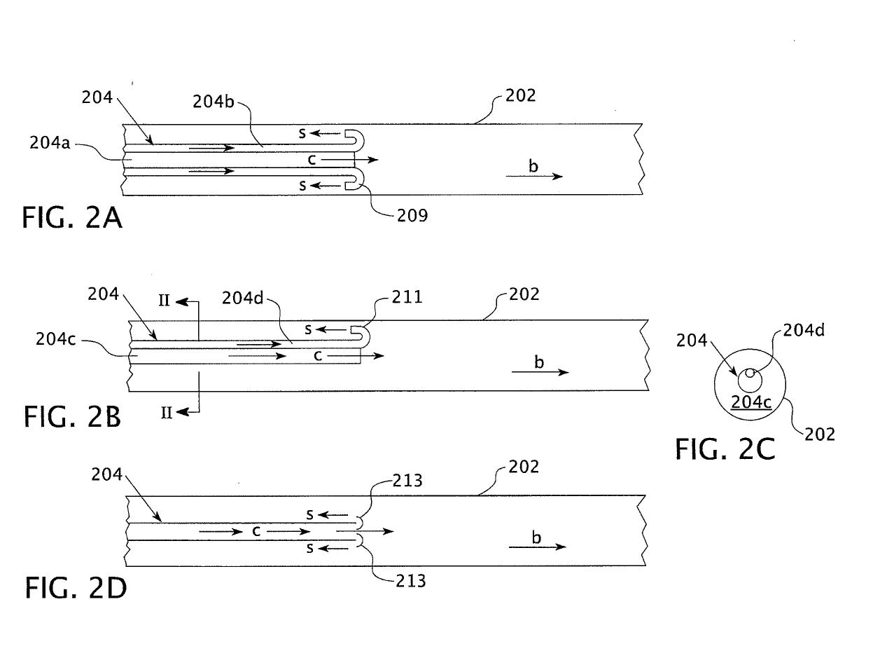

FIG. 2A illustrates, in cross-sectional view within vessel 202, a catheter 204 that includes two co-axial and generally concentric tubular or annular structures. Inner tube 204a is configured for accepting and propagating contrast medium or other fluid (such as medication) to be injected into or towards the “final site” within the patient's body, while outer annular tube 204b is configured for accepting and propagating saline solution by way not only of providing a “flush” but also propelling the catheter 204 forward through the patient's blood vessel in a manner described here below.

As shown, contrast flow c takes place through inner tube 204a. Prior to propagating contrast fluid, however, catheter 204 will preferably be propelled through the vessel 202 to the desired site. As shown, this may be accomplished by propagating fluid (e.g. a flow of saline solutions) through outer annular tube 204b, whereby at a distal end of catheter 204 the annular tube 204b flares so as to turn back about 180 degrees (209. Accordingly, any saline solution or other fluid propagated there through will cause the distal end of catheter 204, and thus catheter 204, itself, to propagate along the direction of blood flow b. Also, during the contrast injection, the rearward flow of saline solution might help create a minor “turbulence” that can assist in the mixing of contrast medium and blood, to optionally allow for a uniform distribution of contrast medium, provide a tighter bolus and/or a saline “chaser” for the contrast.

FIGS. 2B and 2C illustrate an alternative arrangement, with FIG. 2C being an end-on cross-sectional view of the catheter 204 of FIG. 2B. As shown, catheter 204 may include two tubes that are nested but not concentric. Major tube 204c may be configured for carrying the flow c of contrast medium, while minor tube 204d may be configured for carrying the flow s of saline solution. By way of a similar principle as in the embodiment of FIG. 2A, minor tube 204d may be redirected in a 180 degree direction (211) at a distal end of catheter 204 so that saline flow s may have the effect of propagating catheter 204 forward.

FIG. 2D illustrates yet another variant embodiment along the lines of the arrangements shown in FIGS. 2A-2C. Here, catheter 204 may have at a distal end thereof one or more redirecting appurtenances 213 (mounted to catheter 204 itself in a manner not illustrated) that accept contrast or saline flow and redirect the same in a “reverse” direction with respect to the blood flow b, to thereby propel catheter 204 forward. The ratio of forward to reverse thrust is controlled by the size and geometry of the appurtenances 213 and associated openings. Saline is preferably used during positioning of the catheter 204 and contrast is delivered once catheter 204 is in place for imaging.

In one advantageous refinement, the central opening amidst appurtenances 213 can be of a design that opens more widely under sufficient pressure (e.g., so that when contrast is delivered, the opening becomes wider). Alternatively, the central opening could be totally absent such that all flow can is rearwardly directed and forward thrust is maximized. Thus, when contrast is injected in such an instance, the rearward jetflow will efficiently mix the contrast with the blood.

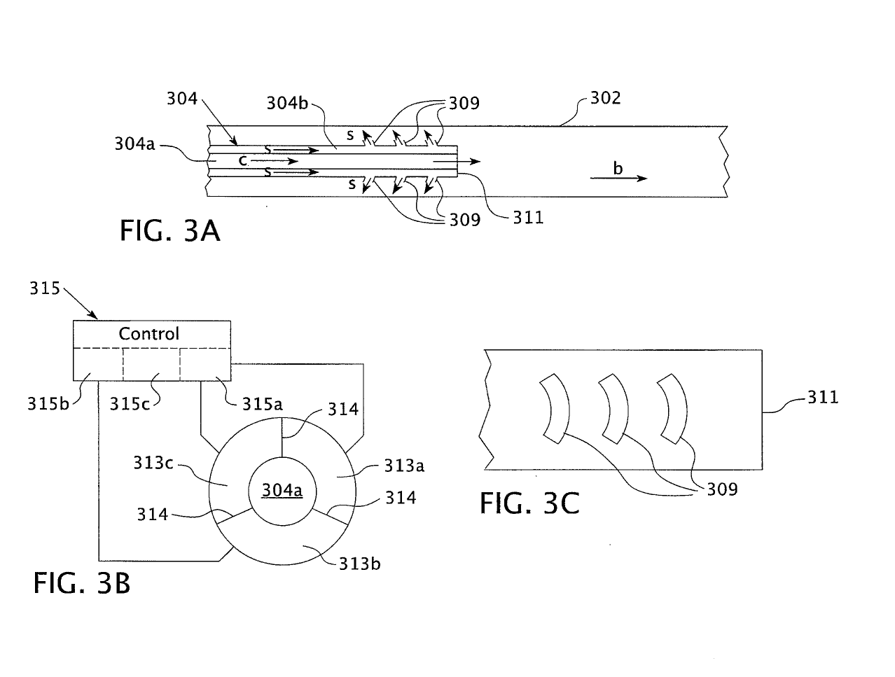

FIGS. 3A-3C illustrate another example of an actively guided catheter in accordance with another embodiment of the present invention. As shown within a vessel 302, catheter 304 may include an inner tubular portion 304a for directing contrast flow c, with an outer annular portion 304b for carrying saline solution flow s. As shown, the outer annular portion 304b may terminate at an end or wall portion 311 at the distal end of catheter 304, thereby compelling saline solution to flow outward through holes or apertures 309.

As shown in FIG. 3C, the holes or apertures 309 may preferably be shaped and oriented such that they promote reverse flow of saline solution to propel catheter 304 forward. Holes 309 may in fact have walls that are specifically angled to promote the desired reverse flow.

As shown in FIG. 3B, outer annular portion 304b may be divided into a plurality of longitudinal chambers (e.g., three in number, 313/a/b/c/), separated by membranes or walls 314. In accordance with a preferred embodiment, saline solution may be administered to these three chambers 313 a/b/c/ separately, governed by a main control 315 with corresponding individual controls 315 a/b/c. In this way, such that by regulating the relative proportions of saline solution propagating through each of the chambers 313 a/b/c, it becomes possible to “steer” the catheter 304, especially if it is necessary to move the catheter through tortuous vessel portions. Pumps that operate two or more syringes simultaneously are well known in the medical arts.

Preferably, catheter 304 may be externally coated with a hydrophilic coating to minimize tissue or vessel trauma, as well as to maintain a lubricious coating for improved mobility.

The inner tubular portion, as shown, is configured to deliver contrast or medication to the region of interest. Preferably, inner tubular portion 304a is centered with respect to the overall structure of catheter 304, but may also be disposed or “biased” towards one side of the catheter 304 or the other. Both tubes 304 a/b and the walls/membranes 314 can be formed via essentially any suitable means, such as extrusion.

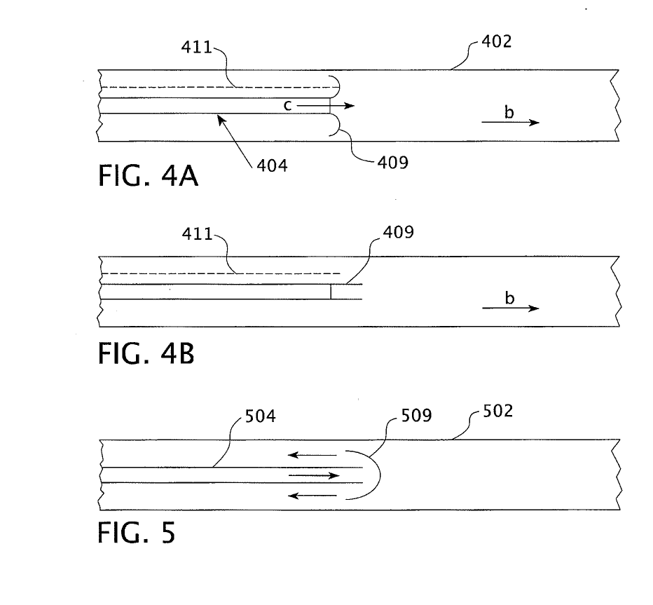

FIGS. 4A and 4B relate to a deployable “umbrella” or “deflector” end on a catheter. As shown in FIG. 4A in vessel 402, distal end of catheter 404 preferably has turned edges 409 when initially deployed in a patient's blood vessel. These turned edges 409, as may be inherently appreciated, will serve to capture and entrain a portion of blood flow b. As shown in FIG. 4B, a suitably configured control wire or tube 411 may preferably be manipulated to transform these edges 409 into a straightened configuration. Preferably, the edges 409 will be formed of a material sufficiently flexible to undergo the transformation as just described, but sufficiently rigid to maintain a consistent “turned” configuration when being deployed.

It should be understood that the embodiment illustrated in FIGS. 4A and 4B can be incorporable into an “actively guided” catheter and thus form a constituent portion thereof. Accordingly, the concept illustrated in FIGS. 4A and 4B can be incorporated, for instance, into the embodiments shown and contemplated in connection with FIGS. 3A-3C, or with FIGS. 2A-2D, or with any other “actively guided” catheter, as may be suitable or viable, discussed or contemplated herein. The embodiment illustrated in FIGS. 4A and 4B could similarly be incorporable into a “passively guided” catheter and form a constituent portion thereof. Thus, the concept illustrated in FIGS. 4A and 4B could be incorporated, for instance, into the embodiments shown and contemplated in connection with FIGS. 1A-1C, or with any other “passively guided” catheter, as may be suitable or viable, discussed or contemplated herein.

In a further variant, a retractable outer sheath could be used to initially cover the edges 409.

FIG. 5 illustrates a catheter 504 (in vessel 502) with a terminal cap or plate 509 which directs fluid in a reverse direction. Here, an injector preferably injects fluid down the catheter 504 as the catheter 504 is inserted through vessel 502. The cap or plate 509 has a rearwardly directed structure as shown to provide reverse fluid flow. In addition, the fluid distends the vein, making insertion easier. The cap or plate 509 does not have to have a significant backward component for there to be a benefit from filling and distending the vein, as mentioned above.

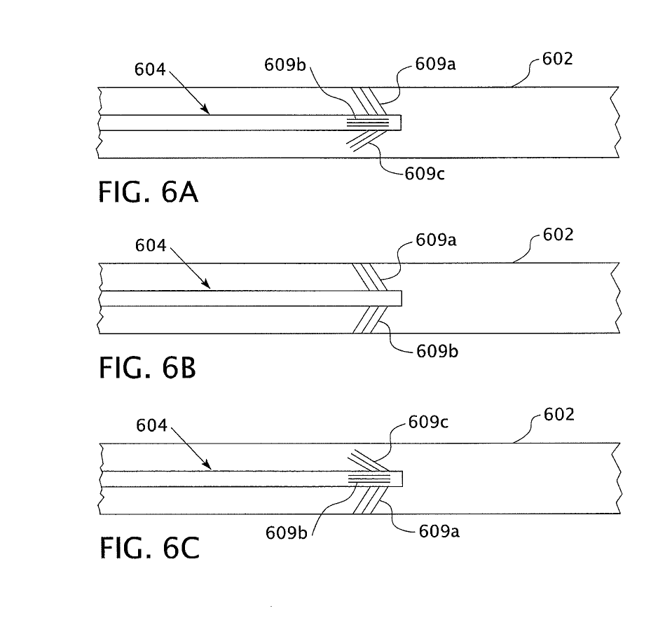

A “rotary millipede” arrangement is contemplated in connection with FIGS. 6A-6C. As shown in vessel 602, catheter 604 may include multiple sets 609 a/b/c of bristles or other soft physical protuberances extending radially away, and in a general proximal direction, from the catheter body. The sets of bristles (e.g., three sets) may be distributed evenly about the circumference of catheter 604 (e.g. 120 degrees apart in the case of 3 sets of bristles). The bristles 609 a/b/c are preferably of such a length as to be extendable to the walls of vessel 602. Each set 609 a/b/c preferably includes subsets of bristles angled clockwise and counter clockwise with respect to the axis of the catheter. Essentially, rotary motion of the catheter 604 pushes it forward in the vessel. This can be understood by looking at FIGS. 6b and 6c. When rotated clockwise in 6b, the bristles that were angled in the clockwise direction have slightly increased friction against the vessel wall and so are straightened out, moving the catheter forward. The bristles angled counter clockwise slide over the vessel wall. When the direction of rotation is reversed, the counter-clockwise angled bristles straighten out, again pushing the catheter forward and the clockwise oriented bristles simply slide over the vessel 602. A similar phenomenon would occur at bristle sets 609b & 609c.

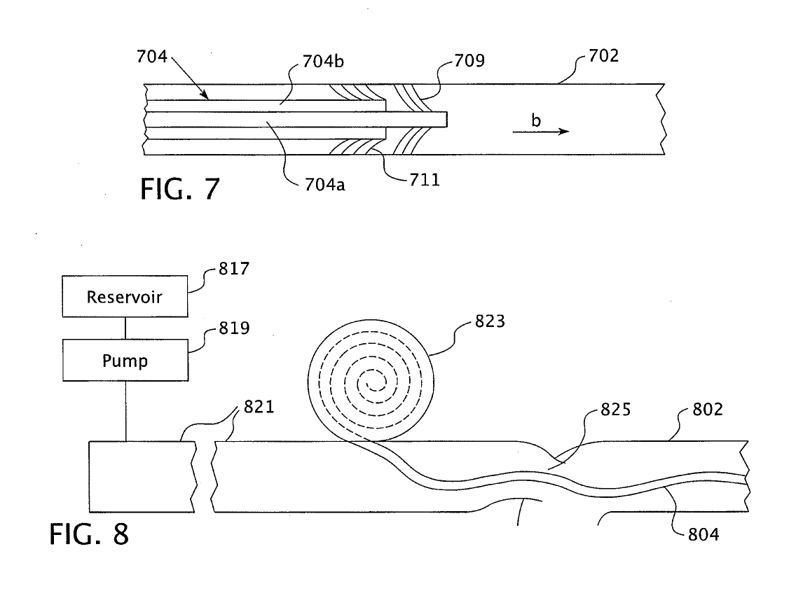

Alternatively, FIG. 7 shows an “axial millipede” arrangement. Here, in vessel 702, an inner member 704 of catheter 704 is slidingly reciprocable with respect to an annular outer member 704b concentric thereto. Inner member 704a and outer member 704b each may have soft bristles or other physical protuberances 709/711, respectively, as shown. Bristles 709/711 are extendable to the inner wall of vessel 702. As such, bristles 711 of outer member 704b can essentially serve as an anchor while inner member 704a moves forward and, likewise, bristles 709 of inner member 704a can serve as an anchor while outer member 704b moves forward. As such, bristles 709/711 are preferably sufficiently rigid as to enable such an anchoring effect, while are also sufficiently soft as to facilitate easy and painless retraction of the entire catheter 704 from a patient. In another variant, the bristles 709/711 may be absent, with the inner member 704a being very flexible with the outer member 704b being stiffer, whereby, the inner member 704a can be propelled forward via blood flow b (perhaps assistively with wings or another “passive” aid).

FIG. 8 illustrates a “pump assisted” system in accordance with another embodiment of the present invention. Preferably, a reservoir (e.g., containing saline solution) and pump 819 will serve to pump saline solution into a vessel 802 via an injecting arrangement 821. A catheter 804, fed from a coil 823 or other sterile storage container that is attached to or integral with injecting arrangement 821, preferably enters a patient through portion 825 of injection arrangement 821. (Portion 825 is inserted into the patient's vessel in a manner similar to a current peripheral IV catheter, optionally a needle-over-catheter arrangement.) It will be appreciated that the incoming flow of saline solution through the narrow segment 825 into the vein will assist in pushing catheter 804 onward.

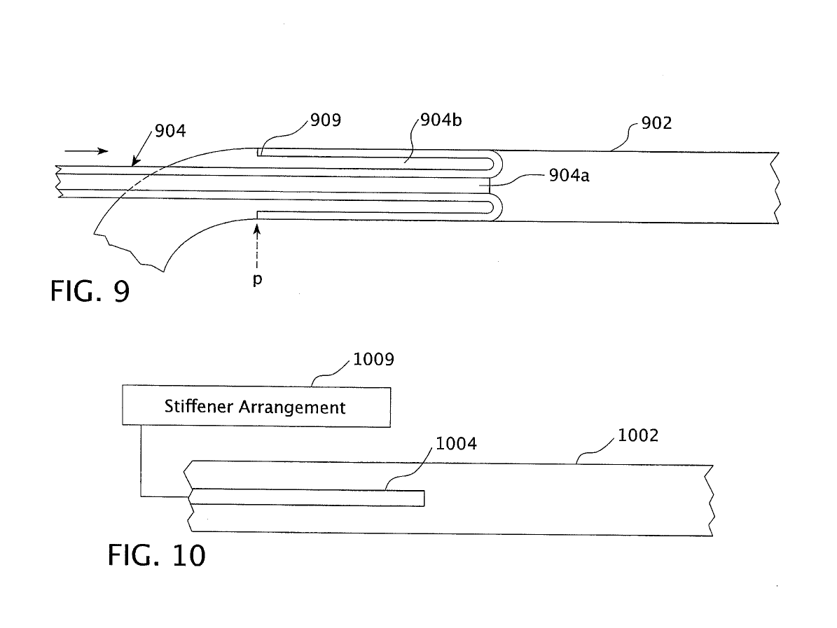

FIG. 9 illustrates an “everting” catheter in accordance with an embodiment of the present invention. As shown, catheter 904 preferably has flexible walls configured for being disposed against walls of vessel 902. An end portion 909 of catheter 904 will thus initially be disposed at vessel wall 902 at point p as indicated, essentially serving as an “anchor”. As the catheter 904 is advanced in the direction of the arrow, catheter 904 will essentially turn inside out so that an increasing length of catheter 904 is disposed against the walls of vessel. In this manner, catheter 904 will easily follow and conform to the natural contours of vessel 902, which would be a huge advantage in the case of particularly tortuous vessels. By lying against the vessel walls, catheter 904 also protects the walls from dissection or abrasion. After delivering fluid (e.g., contrast or medication) through central lumen 904a desired, catheter 902 can be retracted in reverse.

Optionally, the walls of catheter 904 can be formed from a biodegradable material that can be left in the vessel 902 permanently. Used in this manner, it can remain in the vessel 902 to assist in the future delivery of, e.g., drugs, genes, stem cells, or proteins to promote healing in vessels weakened by disease or repeated chemotherapy. In another variant, a biocompatible, but non-degradable “everting” catheter could be used as a permanent implant that would replace damaged blood vessel endothelium, similar to a stent or stent-graft. In this capacity as a prosthetic vessel lining, the catheter wall making contact with the vessel surface could also be coated with e.g., medications, cells, or proteins to promote healing, reduce thrombosis risk, and/or slow the process of diseased vessel wall remodeling. The outside catheter wall could also be coated with non-thrombogenic materials such as heparin to reduce the risk of clot formation.

The eversion phenomenon described above could be “powered” by fluid pressure between the catheter walls (in annular space 904b), in accordance with a further variant. In this case, both ends of the catheter 904 would need to be outside the vein and a sliding seal could be provided at the entry point into vessel 902 Saline solution could be injected between the walls (into annular space 904b), to provide the fluid pressure. Alternatively, the fluid pressure could be minimal but still serve as a physical “buffer” between the catheter walls to reduce friction.

In yet another variant, the everting effect described above could represent a way to push the catheter through the vein, and not form the wall of the catheter itself. In this case, the everting section would fold back shown as the catheter itself is pushed forward, but not touch the wall of vessel over its whole circumference. The everted section could then be withdrawn afterward or left in as part or the totality of the catheter.

FIG. 10 illustrates a “variable stiffness” catheter 1004 in accordance with an embodiment of the present invention. As shown in vessel 1002, a catheter 1004 may be controllably stiffened and/or relaxed via a stiffener arrangement 1009. Through controllable stiffening, great ease could be involved in introducing catheter 1004 into vessel 1002, while a higher stiffness can be maintained as needed to rapidly move through a straight vessel segment, and then be reduced for slow manipulation through tight curvatures encountered in tortuous small vessel branches.

The stiffener arrangement 1009 could take on a wide variety of forms. For instance, it could involve an arrangement for placing the catheter in cold water to stiffen it for insertion, which would then soften at body temperature. Alternatively, the catheter could have a magnetic component (for example, two cross windings that behave like a ferro fluid) which would become more rigid in the presence of a magnetic field that would cause the two cross windings to attract and bind. In another variant, there could be wires in the catheter wall that become stiffer or more flexible in response to externally applied heat or current. Or, the catheter could be constructed with electro-active polymers (EAP's), whose stiffness is related to applied voltage or current. Overall, it will be appreciated that a wide variety of implementations are possible, with the common objective being an arrangement (1009) for controlling the stiffness of catheter 1004 as catheter 1004 is being moved into and through a body, the stiffness being variable to accommodate a variety of prospective conditions.

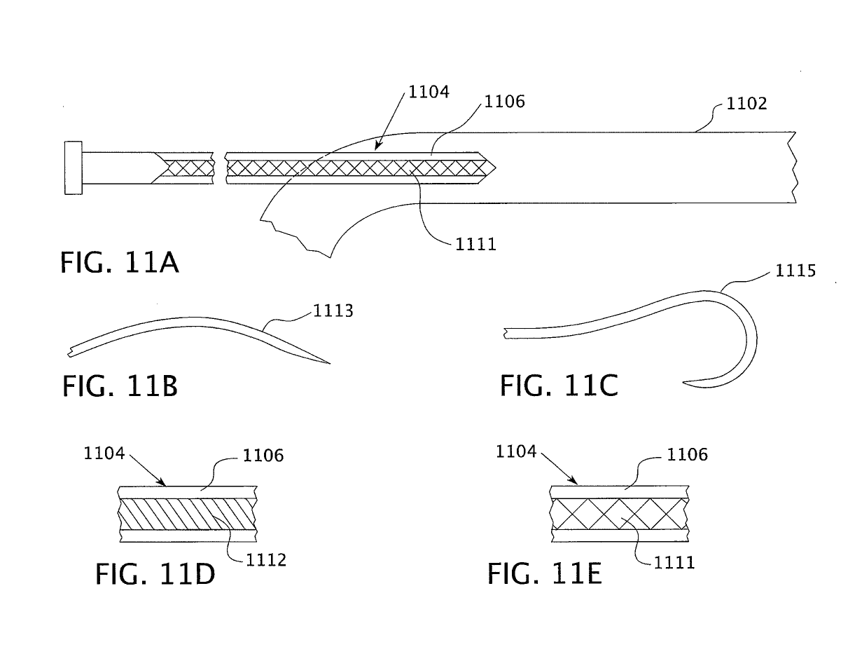

FIG. 11A shows in vessel 1102, a polymer catheter 1104 with an outer sheath 1106. This embodiment can be looked upon as improving upon a conventional torque angiography catheter, and thus is especially well-configured for traversing through tortuous vessels. (While torque catheters are normally designed to navigate through torturous vessels in arterial vasculature, broadly contemplated here is an advantageous arrangement for navigating, at the very least, in tortuous veins).

Catheter 1106 may preferably involve a three-layer construction, including an inner layer of biocompatible polymer (capable of withstanding the forces of braiding, and which resists kinking during use) multiple filament cross-wound metallic or polymer braiding 1111 and a biocompatible polymer overcoat (not illustrated). The cross-wound braiding is preferably configured to provide torque to position the catheter tip to the selected body region.

The outer sheath 1106 could be either over-molded through extrusion technology or applied by way of a shrink-wrap material. Either way may be appropriate for a given application at hand, although extrusion would appear to yield more favorable results. Extrusion materials could be custom-compounded to enhance softness, biocompatibility, and maneuverability. Optionally, a hydrophilic coating could be applied atop outer sheath 1106 to further enhance maneuverability and minimize tissue trauma.

FIGS. 11B and 11C illustrate optional shaped catheter tips 1113 and 1115, respectively, similar to angiography catheters, for providing additional maneuverability for placement.

As shown in FIG. 11D, as an alternative to cross-wound braiding, a single filament 1112 could be wound around the inner tube layer of catheter 1104 to form a continuous coil; this can yield similar torque capability yet allow more inherent flexibility than a cross-wound braid. Particularly, due to a dramatically reduced winding pitch angle, there will be increased flexibility (particularly helpful in the context of less robust venous structure).

FIG. 11E, for its part, provides a close-up view of a portion of catheter 1104 having the cross-braid configuration of FIG. 11. A.

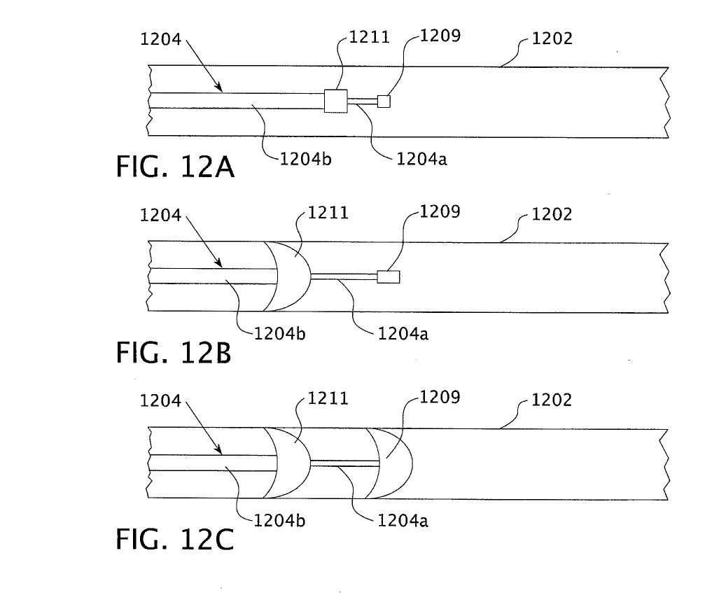

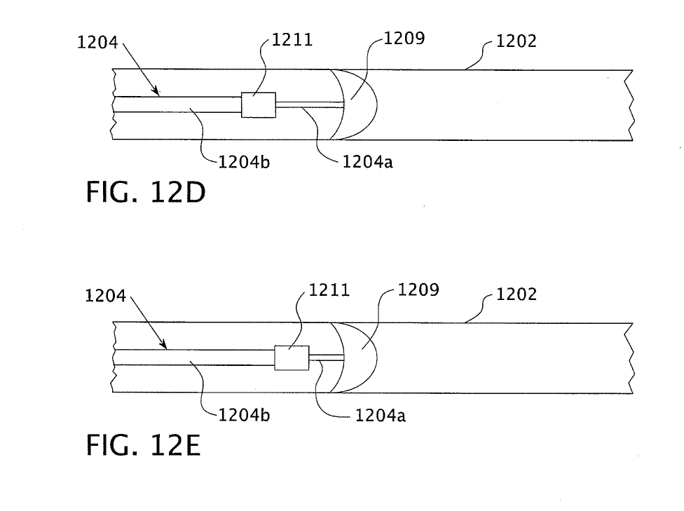

FIGS. 12A-12E illustrate a “walking catheter” in accordance with an embodiment of the present invention. As shown in vessel 1202, catheter 1204 preferably includes two lumens as defined by portions 1204a and 1204b. Inner portion 1204a is preferably formed from elastic and outer annular portion 1204b is preferably formed from a more rigid material. A first balloon 1209 is in fluid communication with inner portion 1204a and a second balloon is in fluid communication with outer portion 1204b. Balloons 1209/1211 are preferably separate from one another and are independently controlled via separate fluid paths.

FIG. 12A shows an initial state where both balloons 1209/1211 are uninflated. To advance catheter 1204, balloon 1211 is preferably inflated as shown in FIG. 12B, e.g. via saline solution. With balloon 1211 now fully inflated and serving as an anchor, further fluid then delivered to balloon 1211 will progress into inner portion 1204a, whereby (uninflated) balloon 1209 is advanced forward as a result. Preferably, the elastic material of inner portion 1204a will be suitably configured to expand not radially but only longitudinally.

At this point, balloon 1209 is preferably inflated as shown in FIG. 12C, to ensure that the same will also now serve as an anchor. Continuing, as shown in FIG. 12D, rear balloon 1211 is preferably deflated which then causes the same to advance toward the front balloon 1209, as shown in FIG. 12E; here, the elastic material of inner portion 1204a is relieved of its tension. Upon then deflating front balloon 1209, the process can restart as in FIG. 12A. Each of the balloons 1209/1211 is independently operated to achieve this repeating sequence by which the entire catheter assembly advances through the vessel 1202.

Optionally, front balloon 1209 could be equipped with a pressure relief valve at is tip to open up beyond a given threshold pressure and permit fluid (e.g., contrast fluid) to progress onward into the vessel 1202. Generally, to preclude occlusion of vessel 1202, balloons 1209/1211 could have a cross-section that permits continued blood flow (e.g., a star-shaped cross-section).

By way of a further alternative, instead of an elastic tube between the balloons 1209/1211, a rolling diaphragm or the like could be provided that would expand longitudinally but not radially. The present invention, in accordance with various additional embodiments, further relates to equipment associated with or supportive of catheters. It should be understood that such equipment, as discussed here below and broadly contemplated herein in general, can be used with essentially any compatible catheter arrangement, including, as appropriate, any or all of the catheter arrangements described and contemplated hereinabove.

As such, the disclosure now turns to various arrangements for maintaining the sterility of a central venous catheter while it is being deployed in a patient.

Normally, if a long catheter such as a PICC needs to be inserted into a vein, it has to be kept sterile before insertion. Typically, this involves draping a significant area of the patient and patient support around the insertion site.

In accordance with an embodiment of the present invention, the catheter line can essentially be kept inside a sterile container or package until it goes into the patient, so that the sterile field need not be much larger than is normally the case for a simple IV catheter (which typically involves just washing around the site).

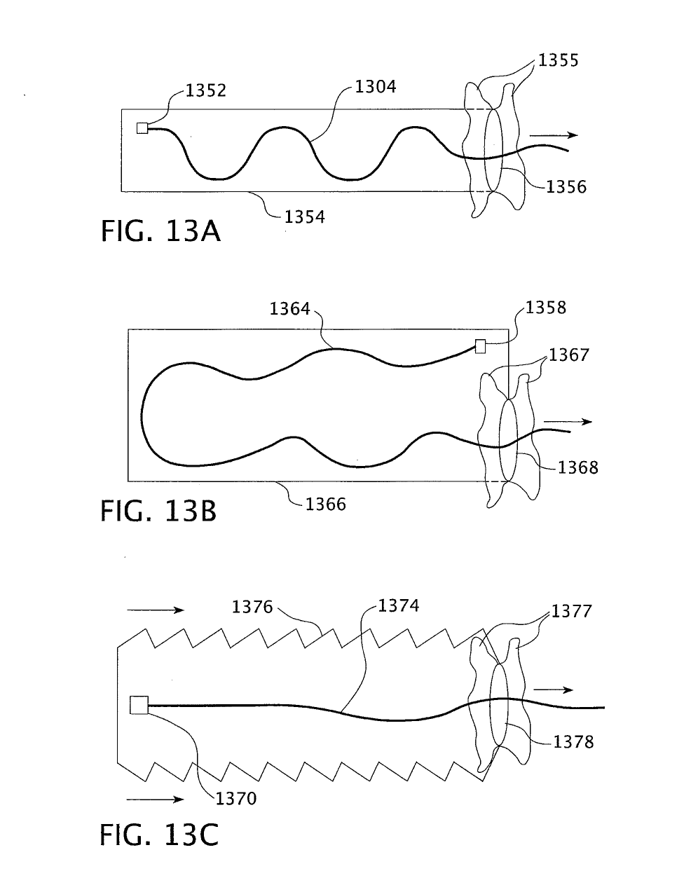

FIG. 13A shows a catheter 1304 extending from hub 1352 in an elongated plastic bag 1354. When the package is opened, flap portion 1355 can surround washed skin to create a sterile field on all sides around the entry point of catheter 1304 into a body. Tape can hold bag 1354 to washed skin at opening 1356.

FIG. 13B shows a less elongated bag where a partial sterile field is created by taping bag 1366 to the skin at flap portion 1367 about opening 1368. The catheter 1368 here extends from hub 1358 and “doubles back” within bag 1366 as shown

In the variants shown in FIGS. 13A and 13B bags 1354/1356 could have some stiffness or rigidity to help support catheter 1304/1364. Alternatively, as shown in FIG. 13C, a bag 1376 (with opening 1378 and flap portion 1377) could crinkle up or gather as the catheter 1374 (extending from hub 1358) is inserted into a patient, while still maintaining a sterile field.

It will be appreciated that with each of the variants shown in FIG. 13A/B/C easy access is afforded to flush and fill the catheter line (e.g., with saline solution) before insertion into a patient if that is deemed to be needed.

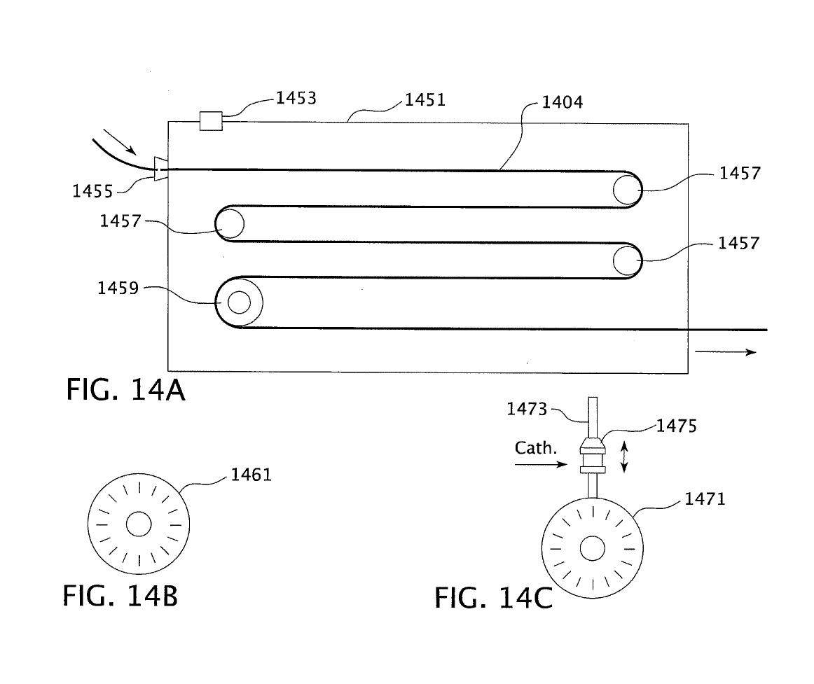

In terms of another type of support equipment for catheters, as shown in FIG. 14A, a dispensing case or bag 1451 may include a luer hub 1455. Hub 1455 preferably accommodates additional lengths of catheter 1404 from the outside as shown by the arrow. A filling valve 1453 may be provided for filling the case or bag 1451 with alcohol or antiseptic. While catheter 1404 is fed along, e.g., guide wheels or hubs 1457, there is also preferably provided a rotary mount 1459 for accommodating a measuring wheel 1461 (see FIG. 14B). Measuring wheel 1461 may thus be selectively mounted on rotary mount 1459, e.g. via a snap fit.

Preferably, measuring wheel 1461 works in the manner of a surveyor wheel, to measure a linear distance to indicate the length of catheter 1402 that is payed out. Alternatively, wheel 1461 could be integrally fixed to mount 1459 initially.

In yet another alternative, wheel 1461 could be completely separate from bag/case 1451 at all times and be used to measure a linear distance along the body of a patient, from the site of catheter insertion to a reasonable approximation of the desired location as “mapped” to the outside of the patient. In this manner, a needed length of catheter can be predetermined and then payed out and/or measured by essentially any suitable means and/or possibly by use of the same or another measuring wheel mounted onto case or bag 1451. If the same measuring wheel is used, it can be used to “count down” a length of catheter fed therepast, starting with the original measurement determined from the outside of the patient's body. Alternatively, one measuring wheel can be used to measure on the patient and a second sterile wheel, used in the package, can be used to measure the catheter length.

FIG. 14C shows another variant. Particularly, a measuring wheel 1471 could be rotatably mounted on a holder 1473 that conveniently is configured to be held by a technician or doctor. Wheel 1471 can be rolled along the outside of a patient's body as just described, to get a close approximation of the length of catheter that subsequently will be needed. The catheter is then preferably fed, along a direction indicated by the horizontal arrow, through a “guillotine” or other cutting device 1475 mounted on holder 1473. Using wheel 1471 to “count down” from the length just determined the catheter 1475 can be cut by cutting device 1475 when the wheel 1471 has reached “zero”. Alternatively, the measuring wheel or some other arrangement could be used to determine the desired length, and the package, for example that of FIG. 13A, could have printed gradations on the outside to allow the catheter to be cut to the desired length before or as it is withdrawn from the package.

An alternative to cutting the catheter to length is to leave the extra length coiled up in a compact arrangement similar to that shown in relation to FIG. 8. This is especially advantageous for contrast injections where the catheter will only be in place for a limited duration.

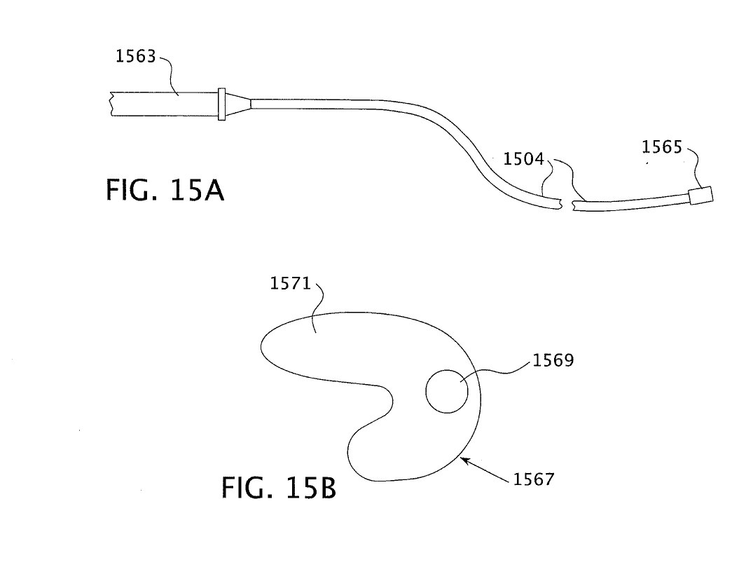

Yet another scheme of catheter support is illustrated in FIGS. 15A and 15B. As shown in FIG. 15A, a catheter 1504 (e.g., a PICC catheter) may extend from an electrical line 1563. At a distal end thereof, catheter 1504 also preferably includes a metal or magnetic tip 1565 which can be powered to activate an outside device for picking up the location of the tip within the patient's body (much like a “stud finder” used in construction applications). Accordingly, the location of tip 1565 within a patient's body could be continually monitored or verified, as could a final position of tip 1565 at a presumed site of interest.

FIG. 15B shows a handheld device 1567 that could be used to locate tip 1565. Preferably, it is embodied by an easily “gripped” object (e.g., wand) that can be passed over a patient's body to find the tip 1565. A light indicator 1569 (e.g. LED) could light up when the tip 1565 is found. A handle 1571 specifically configured for conveniently accommodating a technician or doctor's grip can be provided. As an alternative, tip 1565 could be located by an outside triangulation device such as a GPS.

Generally, catheters, mechanical portions, balloons and other components as described hereinabove and broadly contemplated herein, in accordance with at least one presently preferred embodiment of the present invention, can be formed from essentially any of a very wide variety of plastics, metals or other materials generally used in the medical arts. Examples include, but are by no means limited to, polyurethanes, silicones, teflons, polyethylenes, copolymers, multi-layered structures, nitinol, stainless steel.

Without further analysis, the foregoing will so fully reveal the gist of the embodiments of the present invention that others can, by applying current knowledge, readily adapt it for various applications without omitting features that, from the standpoint of prior art, fairly constitute characteristics of the generic or specific aspects of the embodiments of the present invention.

If not otherwise stated herein, it may be assumed that all components and/or processes described heretofore may, if appropriate, be considered to be interchangeable with similar components and/or processes disclosed elsewhere in the specification, unless an express indication is made to the contrary.

If not otherwise stated herein, any and all patents, patent publications, articles and other printed publications discussed or mentioned herein are hereby incorporated by reference as if set forth in their entirety herein.

It should be appreciated that the apparatus and method of the present invention may be configured and conducted as appropriate for any context at hand. The embodiments described above are to be considered in all respects only as illustrative and not restrictive. All changes which come within the meaning and range of equivalency of the claims are to be embraced within their scope.

1. A catheter comprising:
a catheter body; and
an arrangement for promoting movement of said catheter body within a blood vessel;
said arrangement for promoting movement comprising at least one entraining arrangement for entraining blood flow and urging said catheter body forward within a blood vessel.

2. The catheter according to claim 1, further comprising a retractable sheath for releasing said at least one entraining arrangement.

3. The catheter according to claim 2, wherein said retractable sheath is retractable between:
a first position, wherein said retractable sheath covers said at least one entraining arrangement, whereby said at least one entraining arrangement does not entrain blood flow; and
a second position, wherein said retractable sheath is disposed away from said at least one entraining arrangement, whereby said at least one entraining arrangement entrains blood flow.

4. The catheter according to claim 1, wherein said at least one entraining arrangement comprises a parachute arrangement.

5. The catheter according to claim 1, wherein said at least one entraining arrangement comprises a plurality of wings or flaps extending from said catheter body.

6. The catheter according to claim 1, wherein said at least one entraining arrangement comprises a shroud disposed towards an end of said catheter body.

7. The catheter according to claim 6, wherein said shroud comprises a plurality of perforations to permit blood flow therethrough.

8. The catheter according to claim 1, wherein:
said at least one entraining arrangement comprises at least one deformable edge portion disposed at an end portion of said catheter; and
said catheter further comprises a control which displaces said at least one edge portion between:
a first configuration, wherein said at least one deformable edge portion is oriented to entrain blood flow and urge said catheter body forward within a blood vessel; and
a second configuration, wherein said at least one deformable edge portion is substantially straightened in parallel to a remainder of said catheter.

9. The catheter according to claim 1, wherein said control comprises a control wire.

10. A catheter comprising:
a catheter body; and
an arrangement for promoting movement of said catheter body within a blood vessel;
said arrangement for promoting movement comprising at least one propulsion arrangement for applying a force from outside said catheter to urge said catheter body forward within a blood vessel.

11. The catheter according to claim 10, wherein said at least one propulsion arrangement comprises an arrangement for directing at least one reverse jet flow.

12. The catheter according to claim 11, further comprising:
a first conduit for accepting and directing a fluid; and
a second conduit for accepting and directing a fluid;
said directing arrangement comprising said second conduit.

13. The catheter according to claim 12, wherein said second conduit comprises an exit portion which directs fluid in a direction different from a direction in which said first conduit directs fluid.

14. The catheter according to claim 13, wherein said exit portion of said second conduit directs fluid in a direction offset greater than 90 degrees with respect to a direction in which said first conduit directs fluid.

15. The catheter according to claim 14, wherein said exit portion of said second conduit directs fluid in a direction offset about 180 degrees with respect to a direction in which said first conduit directs fluid.

16. The catheter according to claim 12, wherein said second conduit comprises an annular conduit disposed about said first conduit.

17. The catheter according to claim 16, wherein said exit portion comprises a portion of said second conduit which is turned to direct fluid in a direction different from a direction in which said first conduit directs fluid.

18. The catheter according to claim 17, wherein said exit portion comprises at least one opening for directing fluid in a direction different from a direction in which said first conduit directs fluid.

19. The catheter according to claim 18, wherein said at least one opening is dimensioned to direct fluid at a non-zero angle with respect to a direction in which said first conduit directs fluid.

20. The catheter according to claim 19, wherein said at least one opening comprises a plurality of openings.

21. The catheter according to claim 16, wherein said second conduit is subdivided into a plurality of minor conduits.

22. The catheter according to claim 21, wherein said exit portion comprises, in each of said minor conduits, at least one opening for directing fluid in a direction different from a direction in which said first conduit directs fluid.

23. The catheter according to claim 21, wherein each of said minor conduits is configured for accepting fluid separately to promote selective steering of said catheter body.

24. The catheter according to claim 12, wherein said second conduit is nested within said first conduit.

25. The catheter according to claim 16, wherein said exit portion comprises a portion of said second conduit which is turned to direct fluid in a direction different from a direction in which said first conduit directs fluid.

26. The catheter according to claim 11, wherein said directing arrangement comprises at least one appurtenance for redirecting at least a portion of fluid flow from said catheter in a direction different from a direction in which said catheter directs fluid.

27. The catheter according to claim 26, wherein said at least one appurtenance directs fluid in a direction offset greater than 90 degrees with respect to a direction in which said catheter directs fluid.

28. The catheter according to claim 26, wherein said at least one appurtenance directs fluid in a direction offset about 180 degrees with respect to a direction in which said catheter directs fluid.

29. The catheter according to claim 26, wherein said at least one appurtenance comprises at least one appurtenance disposed at an edge of said catheter at an end portion of said catheter, to redirect a portion of fluid flow from said catheter in a direction different from a direction in which said catheter directs fluid.

30. The catheter according to claim 26, wherein said at least one appurtenance comprises a cap portion disposed at an end portion of said catheter and which covers at least a major portion of an outlet of said catheter.

31. A catheter comprising:
a catheter body; and
an arrangement for promoting movement of said catheter body within a blood vessel;
said arrangement for promoting movement comprising an arrangement for assisting forward movement of said catheter body via physical engagement with a blood vessel.

32. The catheter according to claim 31, wherein said assisting arrangement comprises bristles extending from said catheter body.

33. The catheter according to claim 32, wherein said bristles comprise a plurality of sets of bristles, said sets being spaced apart about a circumference of said catheter body.

34. The catheter according to claim 33, wherein said bristle sets are spaced apart evenly about a circumference of said catheter body.

35. The catheter according to claim 34, wherein said bristle sets comprise three bristle sets spaced about 120 degrees apart from one another about a circumference of said catheter body.

36. The catheter according to claim 32, wherein said bristles are configured to engage one or more walls of a blood vessel.

37. The catheter according to claim 32, whereby rotary motion of said catheter body urges said catheter body forward in a blood vessel.

38. The catheter according to claim 37, wherein some bristles are generally oriented at a different angle with respect to other bristles whereby, as said catheter body undergoes rotary motion, a distal end of said catheter body translates.

39. The catheter according to claim 31, wherein said catheter body comprises an inner member and an outer member, said inner member being slidingly reciprocable with respect to said outer member, said outer member being substantially concentric with respect to said inner member.

40. The catheter according to claim 39, wherein said assisting arrangement comprises bristles extending from each of said inner member and said outer member.

41. The catheter according to claim 40, wherein said bristles of said outer member are configured to anchor said catheter body during sliding movement of said inner member.

42. The catheter according to claim 40, wherein said bristles of said inner member are configured to anchor said catheter body during sliding movement of said outer member.

43. The catheter according to claim 39, wherein said inner member is formed from elastic and said outer member is formed from a rigid material.

44. The catheter according to claim 39, further comprising a first balloon in fluid communication with said inner member and a second balloon in fluid communication with said outer member.

45. The catheter according to claim 44, wherein said first balloon is configured to anchor said catheter body during sliding movement of said outer member.

46. The catheter according to claim 44, wherein said second balloon is configured to anchor said catheter body during sliding movement of said inner member.

47. The catheter according to claim 44, wherein said first and second balloons each have a cross-section, when inflated, configured for precluding occlusion of a blood vessel.

48. The catheter according to claim 39, wherein said inner member is formed from elastic and is configured to expand longitudinally but not radially.

49. The catheter according to claim 31, wherein:
said catheter body comprises an everting wall portion; and
said assisting arrangement comprises said everting wall portion.

50. The catheter according to claim 49, wherein said everting wall portion is configured for being disposed against walls of a blood vessel.

51. The catheter according to claim 50, whereby said everting wall portion is configured such that as said catheter body is advanced in a blood vessel, an increasing length of said everting wall portion is disposed against walls of the blood vessel.

52. The catheter according to claim 49, wherein said everting wall portion is formed from a biodegradable material.

53. The catheter according to claim 49, wherein said everting wall portion is formed from a biocompatible and non-degradable material.

54-92. (canceled)
